(19) United States
(12) Patent Application Publication
(10) Pub. No.: US 2026/0083570 A1
(43) Pub. Date: Mar. 26, 2026

(54) SYSTÈME DE CHIRURGIE ORTHOPÉDIQUE HUMAINE ET PROCÉDÉ DE PLANIFICATION D'IMPLANTATION

(71) Applicant: Lock-In VCF SA, Rolle (CH)

(72) Inventor: Guillaume LACAZE, La Rippe (CH)

(73) Assignee: Lock-In VCF SA, Rolle (CH)

(21) Appl. No.: 19/340,538

(22) Filed: Sep. 25, 2025

(30) Foreign Application Priority Data
Sep. 25, 2024 (EP) 24202387.7

(51) Int. Cl. A61F 2/44; A61F 2/30; A61F 2/46

(52) U.S. Cl. CPC A61F 2/441; A61F 2/4425; A61F 2/4611; A61F 2002/30624; A61F 2002/443; A61F 2002/4633

(57) ABSTRACT

A system and a method for planning human orthopaedic surgery for restoring the volume and/or the geometry of a bone, by expanding at least one expandable bone implant (1) between a folded configuration and a deployed configuration, the implant including a body extending along a longitudinal axis (L) between a proximal end (11) connectable with an implantation instrument (A) for holding the implant and a distal end (12) intended to be firstly inserted into the bone, wherein the system includes a plurality of expandable bone implants (1) and at least one planning support presenting the implants (1) and instrument (A), with reference to at least one standardized classification of the various types of fractures identified, in particular vertebral compression fractures, the planning aid being implementable in the form of a human-machine interface.

The present application relates to the field of surgery, in particular human orthopaedic surgery and notably to the treatment of a collapsed bone structure by restoring the volume (or correcting) and/or the geometry of this bone structure. The present application particularly relates to instruments for installing expandable bone implants for repairing or restoring damaged bone structures, notably in the spine for the treatment (often called “reduction”) of compression fractures, notably vertebral compression fractures (VCF).

In this field, the problem of restoring the volume of bone structure that has collapsed is well known and the literature contains an abundance of solutions using expandable implants capable of transitioning from a folded configuration to a deployed configuration in order to restore the height of the bone structure, preferably in combination with the injection of bone substitute cement, called cement (or bone cement). Many cements are known and they all have the advantage of being injectable in a liquid or viscous state for a certain period of time, then of hardening (by polymerisation) inside the bone structure in order to stabilise it.

Significant problems in this field relate to the expansion of the implant in order to restore the height of the damaged bone tissue as well as to cement leakage, but also to the installation of implants in bones, notably vertebrae. Indeed, instruments need to be available that facilitate implantation and reduce the duration and the difficulty of the task for practitioners as much as possible.

Other recurring problems in orthopaedic surgery relate to invasiveness (i.e., the aim of making the smallest possible incision and of minimising lesions), but also to the deployment ratio in order to obtain a deployed implant that fills the largest possible volume, while having been introduced through the smallest possible passage. Furthermore, this deployment ratio will impact the distribution of forces for correcting the vertebrae: if the deformability is too great, the cement-injection pressure will deform the pouch rather than restore the height.

Within this context, it is understood that many technical problems remain in the field that are associated with the implants, and which are accompanied by problems relating to the implantation instruments, which are generally too numerous and complex to address the significant issues of invasiveness and the ease and the duration of the surgical intervention. Furthermore, the choice of implants as a function of the type of fracture remains difficult, and solutions offering a pragmatic approach to planning surgical interventions are still lacking in the field.

Within this context, an aim of the present invention is to overcome at least some of the disadvantages of the prior art by proposing a system for orthopaedic implants, for restoring a collapsed bone structure.

This goal is achieved by a human orthopedic surgery system for restoring the volume and/or the geometry of a bon by expansion between a folded configuration and a deployed configuration of at least one expandable bone implant comprising a body extending along a longitudinal axis between a proximal end connectable to an implantation instrument for holding the implant and a distal end intended to be inserted first into the bone. This proximal end can be connected to a gripping instrument (known as an implant holder) and is therefore capable of cooperating with the latter by means of physical attachment or connection means, for example, known to those skilled in the art. Nevertheless, certain embodiments provide specific and advantageous attachment means to facilitate the gripping of the implant by an implant holder and, above all, the release of the implant by an L-shaped movement of the implant holder. On the other hand, by being connectable to the instrument, the implant is generally operable for expansion (in particular by injecting a fluid into it and/or by pushing or pulling on an element of the implant), as is widely known in the prior art. Indeed, many systems include expandable implants that can be actuated when mounted on an implant holder that includes an actuating means for expanding the implant (generally a conduit and/or a rod passing through the implant holder to open into a cavity in the implant and/or cooperate with an implant component that allows its expansion, the actuation generally involving a pushing and/or pulling force). The skilled person will therefore understand from reading this application that the implant can be defined without further detail on the instrument and on the actuation, since these are conventional mechanisms in the field and the system comprising the implant and the instrument is of course fully defined, but that the the implant alone is in fact also well defined in its operable nature independently of the instrument and without unnecessary detail on the actuation mechanism (sliding rod, for example), since these are perfectly standard or conventional mechanisms in the field. It is understood in the field of the present application that the term “actuable” implies pushing or pulling (with or without rotation), and the present application thus provides sufficient explanation for the implant to be considered sufficiently clearly defined without additional reference to the instrument enabling its actuation. Conversely, certain embodiments may relate to the instrument itself, through the originality of its elements allowing the implant to be grasped and/or actuated for expansion, and these characteristics then define the instrument independently of the implant, since they do not require any particular details about the implant other than those relating to the function performed by the instrument.

In certain embodiments, this goal is achieved by a human orthopedic surgery system for restoring the volume and/or geometry of a bone by expansion between a folded configuration and a deployed configuration of at least one expandable bone implant comprising a body extending along a longitudinal axis between a proximal end adapted to cooperate with an implantation instrument to hold the implant and a distal end intended to be inserted first into the bone, the system being characterized in that it comprises a plurality of expandable bone implants and at least one implantation instrument corresponding to each of said implants and at least one planning aid presenting said implants and instrument, with reference to at least one standardized classification of the various types of fractures identified, in particular vertebral compression fractures, characterized in that said implants:
extend along a longitudinal axis between a proximal end connectable to an implantation instrument for holding the implant and a distal end intended to be inserted first into the bone;
are deployable from a folded configuration to an unfolded configuration;
are selected from:
a) an implant comprising a hollow body whose wall is formed by a sheet of biocompatible metal alloy, sealed shut between said proximal and distal ends and having, at least in the folded configuration, a plurality of pairs of folds lying on top of each other and wound around the longitudinal axis, said proximal and distal ends respectively comprising a ring providing an entrance to the interior of the hollow body and a base closing the hollow body, said ring and said base being sealed to the folds lying on top of each other and wound around said sheet over the entire periphery of their respective proximal or distal ends, said sheet being plastically deformable to allow the implant to expand from the folded configuration to the deployed configuration when a fluid is injected into the implant;
b) an implant in which at least two faces, for example upper and lower, each comprise a plate (13, 14) for contact with bone tissue and are connected, via at least one hinge, to at least one pair of support arms each oriented in opposite directions towards one of said distal and proximal ends, the implant having a central axis or a housing capable of receiving such an axis extending through a sliding sleeve at the proximal end to a ring or traction sleeve at the distal end to allow the said ends to be brought closer together, causing the support arms to pivot, causing the plates (13, 14) and expansion of the implant, and either:
at least two other sides of said implant are each covered by and secured to at least one sheet of biocompatible metal alloy in a sealed manner, said sheet comprising a plurality of folds laid on top of each other in a folded configuration, said sheet being plastically deformable with a total surface area greater than or equal to the lateral surface area of the implant in the deployed configuration so as to form a sealed compartment capable of receiving a fluid inside the cavity obtained by the expansion of the implant
an envelope formed by a sheet of biocompatible metal alloy, sealed shut on itself and enclosing said implant from the proximal end to the distal end, said sheet having a plurality of pairs of folds lying on top of each other and wound around the longitudinal axis, said proximal and distal ends respectively comprising a ring providing an entrance to the interior of the hollow body and a base closing the hollow body, said ring and said base being secured, in a sealed manner to the folds lying on top of each other and wound around the longitudinal axis of said sheet around the entire periphery of their respective proximal or distal ends, said sheet being plastically deformable to allow the implant to expand from the folded configuration to the deployed configuration when a fluid is injected into the implant;
c) an implant, at least two sides of which, for example the top and bottom, each have at least one plate, each supported by at least two support arms via a respective hinge, an expansion sleeve or ring, at least two expansion arms each comprising a hinge connecting them to one of the ends of one of the plates and a hinge connecting them to an expansion sleeve or ring that can be moved away from a central axis of the implant to exert traction on the plates, via the expansion arms, causing said support arms to pivot, causing the plates to move apart and resulting in controlled expansion of the implant between said collapsed configuration and said expanded configuration, and:
this implant possibly comprising a casing formed by a sheet of biocompatible metal alloy, sealed shut and enclosing said implant from the proximal end to the distal end, said sheet having a plurality of pairs of folds lying on top of each other and wound around the longitudinal axis, said proximal and distal ends comprising, respectively, a ring providing an entrance to the interior of the hollow body and a base closing the hollow body, said ring and said base being sealed to the folds lying on top of each other and wound around the longitudinal axis of said sheet around the entire periphery of their respective proximal or distal ends, said sheet being plastically deformable to allow the implant to expand from the folded configuration to the deployed configuration when a fluid is injected into the implant.

According to another feature, said planning support is implemented by means of computer technology comprising a human-machine interface and executing instructions on a processor enabling:
the display of information relating to said standardized classification of the various types of fractures identified, in particular vertebral compression fractures;
the selection of at least one type of fracture and at least one piece of information relating to the dimensions of the fractured bone, then
the display of implants and instruments of the system that are usable for the intended restoration and, if necessary, instructions or advice on the procedure to be followed, for example with a display of references for implants or various systems that can be used for each fracture identified in the classification, then
the user selecting one of these proposals and recommendations via the human-machine interface, causing the following instructions or recommendations to be displayed to provide assistance with the procedure or its planning, with the acquisition of data provided by the user on the planned surgical procedure.

According to another feature, the system is characterized in that it comprises at least one expandable bone implant and at least one corresponding implantation instrument, selected from a plurality of implants and instruments available through computer means comprising a human-machine interface and executing instructions on a processor enabling:
display information relating to at least one standardized classification of the various types of fractures identified, in particular vertebral compression fractures;
the selection of at least one type of fracture and at least one piece of information relating to the dimensions of the fractured bone, then
the display of the implants and instruments of the system that are usable for the intended restoration and, where applicable, instructions or advice on the procedure to be followed, for example with a display of references for the implants or various systems that can be used for each fracture identified in the classification, then
the user selecting one of these proposals and recommendations via the human-machine interface, causing the following instructions or recommendations to be displayed to provide assistance with the procedure or its planning, with the acquisition of data provided by the user on the planned surgical procedure.

According to another feature, the system is characterized in that it comprises at least one expandable bone implant and at least one corresponding implantation instrument, selected from a plurality of implants and instruments available through computer means comprising a human-machine interface and executing instructions on a processor enabling:
displaying information relating to at least one standardized classification of the various types of fractures identified, in particular vertebral compression fractures;
selection of at least one type of fracture and at least one piece of information relating to the dimensions of the fractured bone, then
display of the implants and instruments in the system that can be used for the planned restoration and, where applicable, instructions or advice on the procedure to be followed, for example with a display of references for the implants or various systems that can be used for each fracture identified in the classification, then
selection of a choice from among these proposals and advice by the user via the human-machine interface, causing the following instructions or advice to be displayed to provide assistance with the procedure or its planning, with the acquisition of data provided by the user on the planned surgical procedure.

According to another feature, the selection of the fracture type in the classification is performed either by the user or automatically by computer means using training on fracture databases for their automatic classification based on technical information and/or images provided by the user and/or previously acquired by computer means, then correlating them with the standardized fracture classification, the automatic selection preferably being validatable or modifiable by the user.

According to another feature, one of the selectable implants is an expandable bone implant for human orthopedic surgery for restoring the volume and/or geometry of a bone, by expansion between a collapsed configuration and an expanded configuration, said implant comprising a hollow body extending along a longitudinal axis between a proximal end connectable to an implantation instrument for holding the implant and a distal end intended to be inserted first into the bone, and which is characterized in that:
the wall of said hollow body is formed by a sheet of biocompatible metal alloy, sealed shut between said proximal and distal ends;
said sheet has, at least in the folded configuration, a plurality of pairs of folds, each pair comprising an antiform fold, known as convex, and a synform fold, known as concave, said folds lying on top of each other in the folded configuration so that the surfaces between each of said convex and concave folds are wrapped around the longitudinal axis,
said proximal end comprises a ring sealed to the folds lying and wrapped around said sheet over the entire periphery of the distal end, the opening through the ring providing an entrance to the interior of the hollow body of the implant,
said distal end comprises a base closing the distal end and sealed to the flat and rolled folds of said sheet around the entire periphery of the distal end of said hollow body
said sheet being plastically deformable to allow the implant to expand from the folded configuration to the deployed configuration when a fluid is injected into the implant.

According to another feature, one of the selectable implants is an expandable bone implant for human orthopedic surgery for restoring the volume and/or geometry of a bone, by expanding between a collapsed configuration and an expanded configuration, said implant extending along a longitudinal axis between a proximal end connectable to an implantation instrument for holding the implant and a distal end intended to be inserted first into the bone, at least two faces of the implant, for example upper and lower, each comprising a plate for contact with bone tissue, each of the plates comprising a central portion connected, by means of at least one hinge, to at least one pair of support arms each oriented in opposite directions within each pair, one arm of each pair being hinged to the distal end while the other arm is hinged to the proximal end, the implant being adapted to receive or comprising a central shaft extending through a sliding sleeve at the proximal end to a traction ring or sleeve at the distal end where it is adapted to transmit traction, when actuated by an instrument, on the distal end to allow it to be brought closer to the proximal end, causing the support arms to pivot, causing the plates to move away from each other and, consequently, the implant to expand between the collapsed configuration and the deployed configuration.

According to another feature, the implant is also characterized in that:
at least two other faces of the implant, between those comprising the plates, are covered with at least one sheet per face, made of a biocompatible metal alloy, and sealed to the central portions under the plates, to the lateral faces of the arms, and to the lateral faces of the proximal end and the distal end,
said sheet is plastically deformable to allow expansion of the implant and has, at least in the folded configuration, a plurality of anti-form folds, known as convex, and synform folds, known as concave, said folds lying on top of each other in the folded configuration, the total surface area of said sheet being greater than or equal to the lateral surface area of the implant in the deployed configuration so as to form a sealed compartment capable of receiving a fluid inside the cavity obtained by the expansion of the implant.

According to another feature, the implant comprises a casing enclosing the implant from the proximal end to the distal end, and in that:
the casing is formed by a sheet of biocompatible metal alloy, sealed on itself;
said sheet has, at least in the folded configuration, a plurality of pairs of folds, each pair comprising a convex fold and a concave fold, said folds being laid on top of each other in the folded configuration so that the surfaces between each of said convex and concave folds are wrapped around the longitudinal axis;
said proximal end extends into a sealing sleeve that is sealed to the folds lying and wrapped around said sheet over the entire periphery of the proximal end;
said distal end extends into a sleeve that is sealed to the laid and rolled folds of said sheet around the entire periphery of the distal end of said implant;
said sheet is plastically deformable to allow the implant to expand from the folded configuration to the deployed configuration, forming a sealed envelope enclosing the implant and preventing leaks when a fluid is injected inside the implant and the envelope.

According to another feature, one of the selectable implants is an expandable bone implant for human orthopedic surgery for restoring the volume and/or geometry of a bone by expanding between a collapsed configuration and an expanded configuration, said implant comprising a central axis and extending along a longitudinal axis between a proximal end connectable to an implantation instrument for holding the implant and a distal end intended to be inserted first into the bone, at least two faces, for example upper and lower, of the implant each comprising at least one plate for contact with bone tissue, each of the plates being supported by at least two support arms each, via a hinge on the central axis and a hinge under the respective plate of each of said support arms, characterized in that:
an expansion sleeve or ring disposed in the same axis as said central axis;
at least two expansion arms each comprise a hinge connecting them to one of the ends of one of the plates and a hinge connecting them to said expansion sleeve or ring;
said expansion sleeve or ring and the proximal end of the central axis are capable of cooperating, respectively or inversely, with a hollow tube for gripping the implant of an implantation instrument and with an expansion rod of said instrument;
said expansion rod is capable of sliding inside said hollow tube, so as to allow said sleeve and said central axis to be moved apart, exerting traction on the plates via the expansion arms, which causes the support arms to pivot, causing the plates to move away from the central axis, resulting in controlled expansion of the implant between the folded configuration and the deployed configuration.

According to another feature, the implant comprises a sheath enclosing said implant from the proximal end to the distal end, and in that:
said sheath is formed by a biocompatible metal alloy sheet, sealed closed on itself;
said sheet has, at least in the folded configuration, a plurality of pairs of folds, each pair comprising a convex fold and a concave fold, said folds being laid on top of each other in the folded configuration so that the surfaces between each of said convex and concave folds are wrapped around the longitudinal axis;
said proximal end extends into a sealing sleeve that is sealed to the folds lying and wrapped around said sheet over the entire periphery of the proximal end;
said distal end extends into a sleeve that is sealed to the laid and rolled folds of said sheet around the entire periphery of the distal end of said implant;
said sheet is plastically deformable to allow the implant to expand from the folded configuration to the deployed configuration, forming a sealed envelope enclosing the implant and preventing leaks when a fluid is injected inside the implant and the envelope.

Another purpose of this application is to overcome at least some of the disadvantages of the prior art by proposing a method for planning surgical procedures that is easy to use and allows for effective stabilization of bone tissue or even assistance with the procedure and/or monitoring of healing.

This objective is achieved by a method for planning the implantation of an implant system according to one of the preceding claims, for restoring the volume and/or geometry of a bone, by expansion between a collapsed configuration and an expanded configuration, characterized in that it comprises a selection, automatically and/or by the practitioner performing the implantation, of at least one type of fracture identified in at least one standardized classification, via computer means and presented to said practitioner, then access, following this selection, to at least one implant system compatible with said type of fracture and to at least one surgical protocol comprising a series of steps to be performed according to said selection.

According to another feature, the method is implemented by computer means executing instructions on a processor enabling:
the display of information relating to at least one classification of fracture types identified in traumatology;
the selection of at least one fracture type and at least one piece of information relating to the dimensions of the fractured bone, then
display instructions or advice on the procedure to be followed and on the various systems to be used for the procedure, for example with a display of references to implants or various systems that can be used for each fracture identified in the classification, then
select a choice from among these pieces of advice, causing the following instructions or advice to be displayed to provide said step-by-step assistance.

Another feature is that the display of instructions or advice includes the display of selectable functions for choosing between actions to be performed in order to continue the procedure and/or between implants to be selected according to the surgical protocol chosen based on the type of fracture identified according to the aforementioned classification, allowing the practitioner to validate a choice and possibly propose predefined options in the validated protocol, such as acquiring X-rays at the end of certain steps implemented.

According to another feature, the selectable functions include functions that allow the practitioner to modify the protocol and generate the display of a protocol modification window, either to select a protocol from among other possible protocols for the fracture type identified according to the classification, or to create a customized surgical protocol for the fracture being repaired.

According to another feature, said selectable functions allow for the recording of surgical procedures and protocols, particularly customized ones, used by different practitioners and their associations with fractures, with the possible recording of customized protocols created by practitioners, in order to offer them at a later date.

Further features and advantages of the present invention will become more clearly apparent upon reading the following description of various embodiments, with reference to the appended drawings, in which:

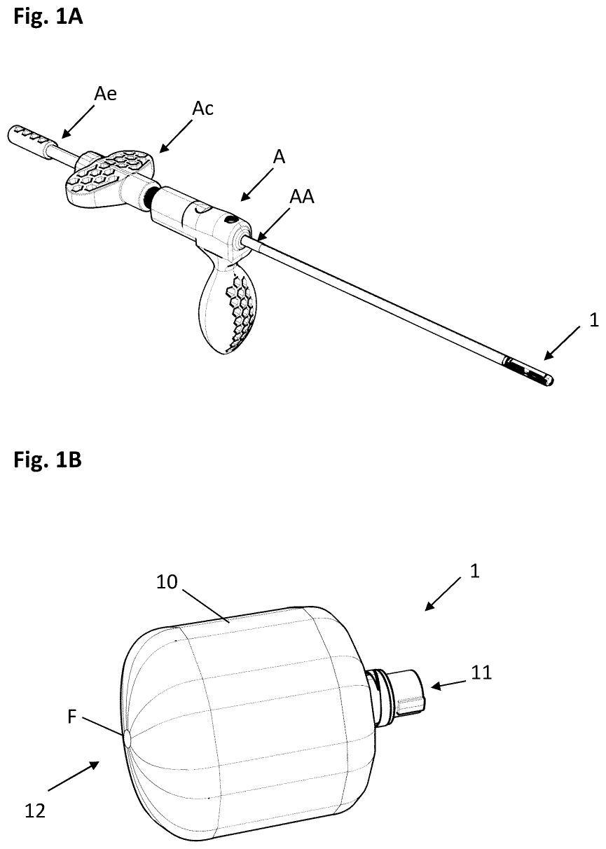

FIG. 1A shows a perspective view of an implantation instrument supporting an expandable bone implant in a folded configuration, according to some embodiments, and

FIG. 1B shows the same implant in a deployed configuration;

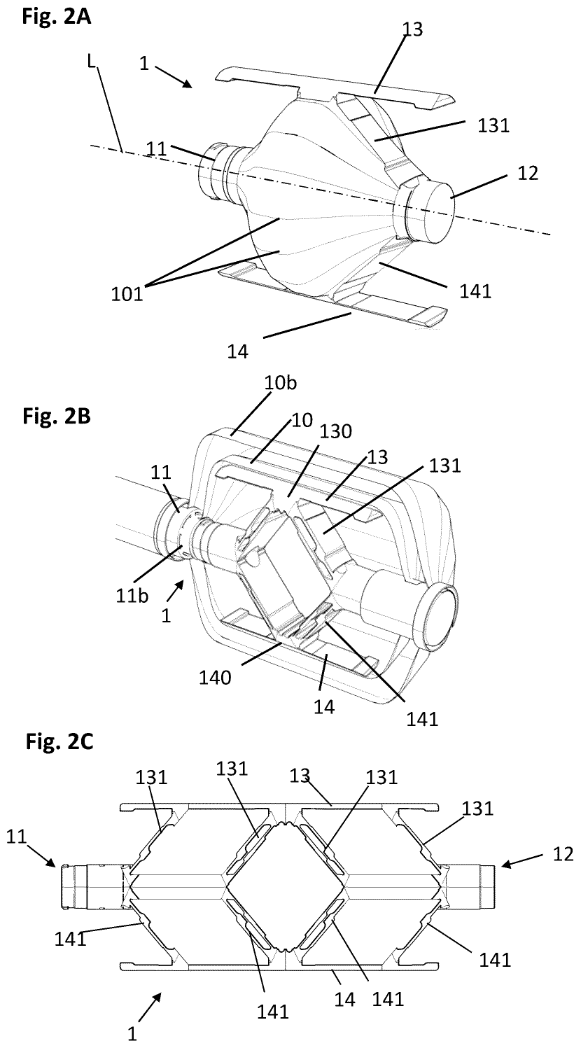

FIG. 2A shows a perspective view of an expandable implant according to some embodiments, FIG. 2B shows a perspective view with a cross-sectional portion of an expandable implant according to some embodiments, and FIG. 2C shows a profile view of an expandable implant according to yet other embodiments;

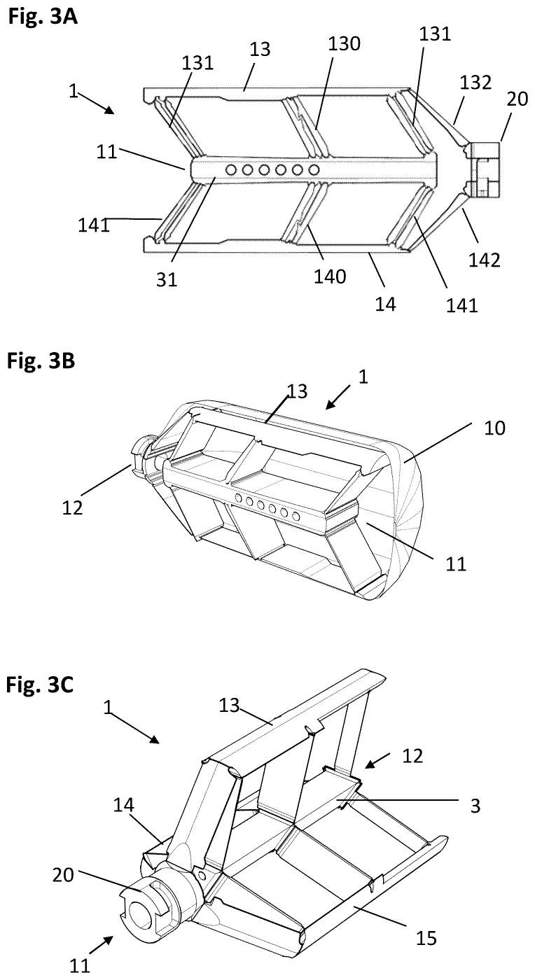

FIG. 3A shows a profile view of an expandable implant according to some embodiments, FIG. 3B shows a perspective view with a cross-sectional portion of an expandable implant according to other embodiments, and FIG. 3C shows a profile view of an expandable implant according to yet other embodiments;

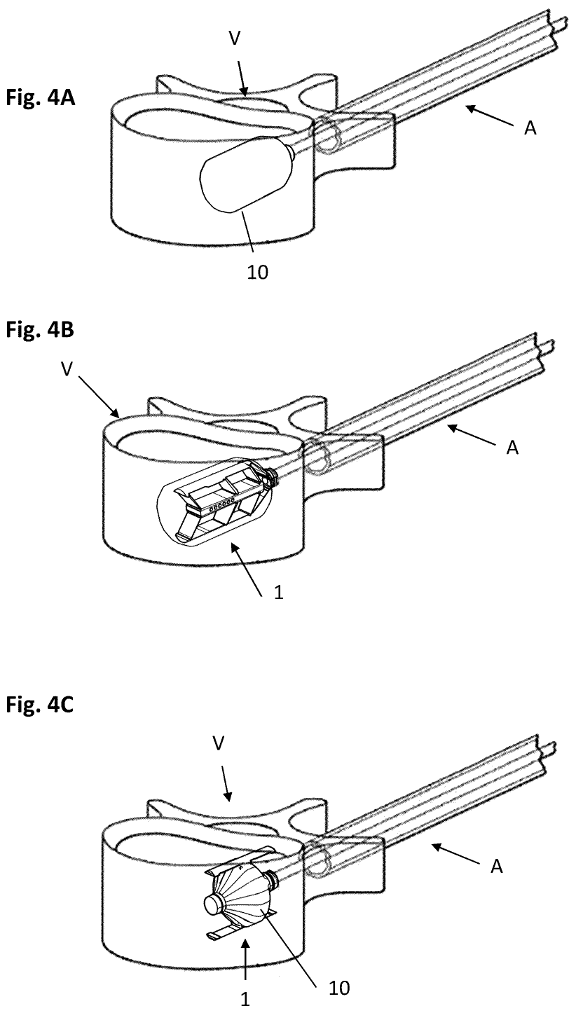

FIG. 4A shows a perspective view of the instrumentation for implanting an expandable implant comprising an implant holder, a cement injection instrument and a gripping instrument, FIG. 4B shows details of the cylindrical section indicated in FIGS. 4A, 4B and 4C show perspective views of vertebrae into which an expandable implant according to various embodiments is inserted;

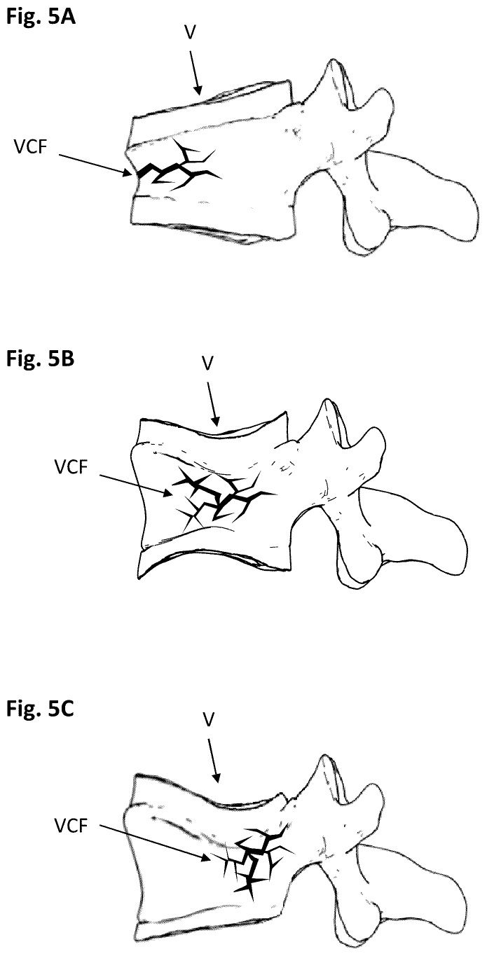

FIGS. 5A, 5B and 5C show profile views of vertebrae that have respectively suffered anterior, median and posterior vertebral compression fractures (VCF);

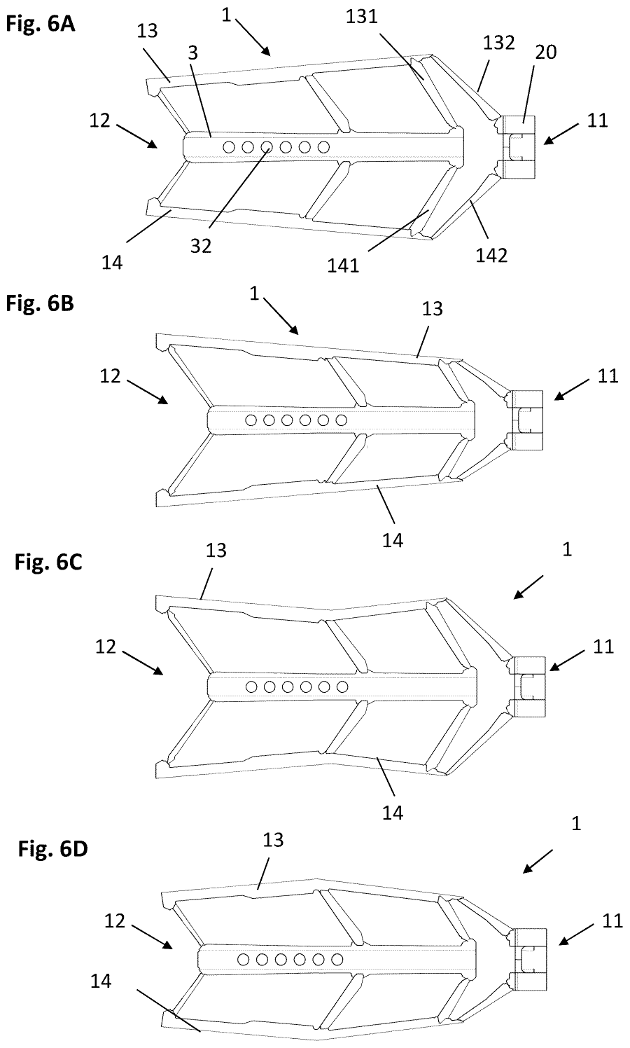

FIGS. 6A, 6B, 6C and 6D show profile views of expandable implants of the same type but with different geometries for adapting to vertebral fractures, notably those shown in FIGS. 5A, 5B and 5C;

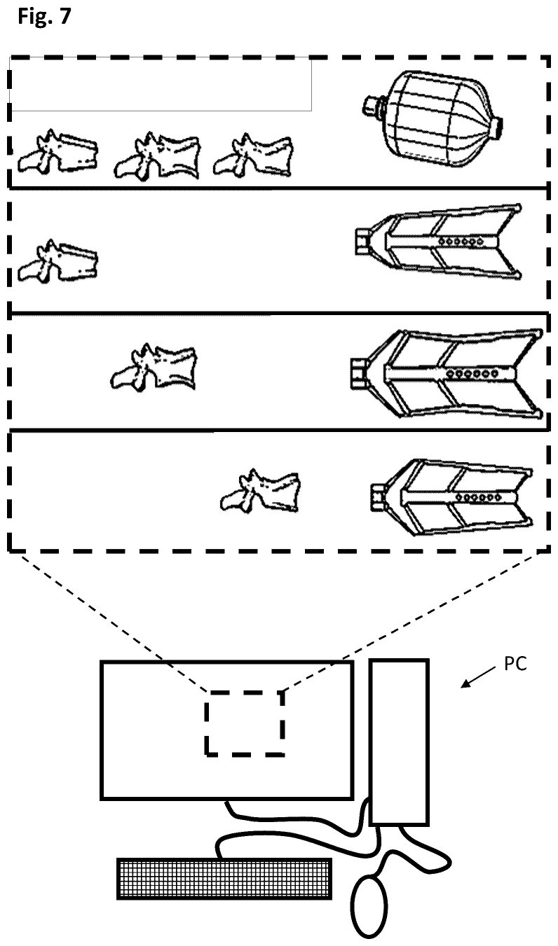

FIG. 7 shows a schematic view of the system for planning an intervention using a display of the fracture classification and implants that can be used in each case.

The present application relates to a system for human orthopaedic surgery for treating bone fractures and bone tissues in general, and to a method for planning a surgical intervention and/or for assisting the intervention. The bone implant is preferably a spinal implant, and in particular a vertebral or even in fact an intervertebral implant, although other uses can be contemplated elsewhere in the spine (intervertebral discs) or in other bone structures where a space left vacant as the result of a fracture needs to be filled (which can be the result of multiple causes, even though they generally involve a reduction in bone density). Thus, vertebral compression fractures (VCFs) are a preferred application but are not the only conditions that can be treated using the present invention, and a person skilled in the art will appreciate the possibilities that are offered without requiring further details herein. In terms of other bones, the femur or the humerus (head of) can be cited, for example, in the event of a risk of collapse. In addition, the tibial plateau is frequently subject to crushing, and the implants or systems of the present application are useful for restoring height in any type of bone crushing or collapse, for example in the distal part of the humerus or femur. On the other hand, as taught, for example, in document EP2921142, it is possible to use expandable implants as bone anchoring implants, and such use is also possible for implants such as those of the present application. In this case, the implants will be extended at their proximal end by an elongated body to which another orthopedic implant of another type or a surgical device for fixing other elements can be attached. Nevertheless, in the case of use as a bone anchor in a vascularized structure, such as a humeral or femoral head, the size of the implant will preferably be limited in relation to the bone structure in order to preserve vascularization and promote bone healing.

Some embodiments comprising more than two flanges notably can prove to be more effective in the treatment of long bones of this type by distributing the expansion forces over more than two surfaces, thereby providing better stability irrespective of the type of bone.

Some embodiments anticipate the injection of a fluid (for example, “bone cement”, generally based on a polymer such as PMMA, for example, and well known to a person skilled in the art, such that no details concerning the cement will be provided herein). Thus, once positioned, the implant notably can be stabilised by such an injection of cement. However, because cement leakages are still a significant problem in this field, various embodiments propose containing the cement in a fluidtight casing, the post-injection volume of which can be controlled by virtue of the structure and the material of the casing, as a function of the injected pressure (and the configuration of the bone tissues, preferably assessed in advance, as is the general practice in this field). Of course, fluidtightness is relative and this term is not limiting either, since the level of fluidtightness is actually adapted to the viscosity of the cement when it is injected. Some embodiments particularly allow proportional expansion of the casing by virtue of the (relative) flexibility of the sheet (10) of biocompatible metallic material. This material is generally a titanium alloy that is obtained in the form of a very thin sheet, preferably by rolling in order to yield a controlled surface condition and a controlled thickness, notably a thickness ranging between 3 and 100 microns, generally between 6 and 50 microns and preferably 10 and 30 microns. In general, the present invention uses at least one sheet (10) of biocompatible metal or biocompatible metal alloy, such as titanium or its alloys, particularly with nickel or others, but also nitinol or stainless steel or their alloys. Advantage is taken of recent techniques for obtaining very thin sheets of such metals, in particular with a thickness of less than 50 or even 40 μm, which makes it possible to obtain relatively flexible and elastic sheets, but above all, the plastic deformation can be used reversibly without reaching their tear limit, by creating folds arranged longitudinally on the implant. In particular, it is possible to provide a maximum deployed volume that is greater than the volume required for the desired applications, so that this limit is never reached and it is possible to fold and then redeploy the implant, even several times (for example, in the event of incorrect positioning of the implant) without the risk of uncontrolled tearing and leakage. Thus, thanks to this type of sheet and the configuration of their interlocking folds, it is possible to obtain expansion ratios between the folded volume and the deployed volume ranging from 2 to 20, or even 30, and it is also possible to control the shape of the implant in its deployed configuration, depending on the arrangement of the folds, in the manner of origami. Finally, although one of the main goals here is to prevent cement leakage, it can sometimes be useful to control the release of cement outside the implant, so that we no longer talk about leakage but rather controlled release, for example to allow adhesion to certain surrounding structures (usually bone structures). Similarly, as the injected fluid is not necessarily cement (or at least not the fluid that would come out of the implant), it may in fact be useful to administer molecules through such controlled release of this fluid. Thus, various embodiments provide for a certain porosity of the sheets (10) at least in certain portions of the implant, for example through holes of controlled microscopic size and controlled number and density. In any case, this type of sheet is capable of reversible plastic deformation for a number of times that is satisfactory for the target application, since it notably offers the possibility of retracting the casing formed by the sheet in the event of a problem (biocompatibility and resistance to tearing). Indeed, in general, controlling the metering of the cement allows the fifteen minutes of polymerisation time to be monitored, during which time it is possible to retract the casing and aspirate the cement. Furthermore, through the injection of cement and the expansion of the casing the implant fills the spaces in the damaged tissues as a function of the compression and bone-resistance forces relative to the hydraulic pressure supplied during the injection of cement. A closed structure needs to be obtained from such a sheet, which already means that the sheet needs to be closed on itself and locked in position. To this end, welding (or bonding or brazing, with these terms being non-limiting herein) can be used to join together two superposed edges or edges with interlocking turnups, in order to facilitate the welding and make it more robust. Some embodiments therefore contemplate closure by welding from the outside, which is simpler and more robust because of the superposition of layers in the vicinity of these complementary folds.

It should also be noted that the number of folds is not limiting either and that it allows the irregularity or actual shape of the implant to be maintained during deployment, which also has advantages, particularly in terms of stabilization. Furthermore, it is preferable to provide for an even distribution of surface area between the folds to allow for uniform deployment, but the invention also envisages other applications, in particular folds of different sizes depending on the region of the implant, in order to achieve asymmetric deployment and better therapeutic results. Furthermore, the present invention makes it possible to control the shape of the implant once deployed by also providing for the distance between the folds. Indeed, the distance between the synform/antiform folds and therefore the distance between the long folds and the short folds determines the way in which the implant deploys. Advantageously, if the density is higher at a point on the periphery, the deployment will be greater, and if it is lower, the envelope will be able to deploy less. It is understood that this results in asymmetry and curvature through more extensive deployment in the areas with the most folds. Similarly, it is possible to provide more material (a large surface area of the sheet on one side, for example) so that the lateral expansion is greater on that side than on the other. Furthermore, in some embodiments, the sheet is welded to the plates and therefore cannot expand further than the distance between the plates, which will have been set by the lifting mechanism. In certain embodiments, the number of pairs of folds is between 3 and 16, generally 4 to 2, preferably around 8. However, 3 folds may sometimes be sufficient, but the greater the number of folds, the less the material will deform, the less risk there will be of tearing, and the easier it will be to unfold. Thus, it is possible to provide up to 20 folds. In general, it is understood that the implant will retain, even in the deployed configuration, at least some of the folds of the sheet near the proximal and distal ends, but the dimensions and strength properties of the sheet (10) used allow the implant to be obtained and these persistent folds do not interfere with function and do not cause mechanical or physiological problems in the bone tissue. In certain embodiments, the implant comprises, in the deployed position, a middle portion between its two ends which has a generalized cylindrical shape with a length greater than or equal to that of the plateaus, with possible partial persistence of said folds, said middle portion extending, on the proximal end side (11), by a truncated cone portion connecting the middle portion to the sleeve and, on the distal end side (12), by a truncated cone portion connecting the middle portion to the socket, the truncated cone portions having a permanent persistence of at least a portion of folds lying flat and rolled up near the proximal (11) and distal (12) ends. In certain embodiments, said sheet is also plastically deformable from the deployed configuration to the folded configuration, without tearing of the sheet due to the thinness of the sheet and in particular due to the persistence of the folds lying and rolled up at the proximal and distal ends, facilitating the reversibility of the expansion. This results in an implant whose expansion is limited in one dimension (generally the essential dimension, where a specific height or width is to be restored), but not in another dimension, so that the injection of cement will expand the envelope into any areas of low bone density that may be present around the implant. It should also be noted that the fluid injection instrument (Ac) may be equipped with means for controlling the injected pressure (a pressure gauge, for example) and for determining the resulting volume, in order to effectively control expansion in the bone tissue. Finally, it is understood that the instrumentation proposed in this application in certain embodiments, using a relatively conventional implant holder (or ancillary) to hold the implant and insert it into the bone tissue, but also less conventional for expanding it into the bone tissue, also has the advantage of being able to perform all the steps of implantation and stabilization with a single instrument and in a continuous operation. Indeed, the ancillary device with a hollow tube for conveying the cement through the tube that holds the cement provides an instrument that allows the surgical operation to be performed quickly and efficiently. After drilling, the implant is inserted and, without removing the instrument, the envelope can be filled with cement and then the tool can be removed before, during, or even after the cement has polymerized (for example, using a mechanism to cut the hard cement when the instrument is rotated). The duration of the surgical procedure is significantly reduced, of course, but so is the stability of the implant, which is not released at any time until it is stabilized by the injection of cement filling all the free spaces around it, unlike certain prior art solutions.

In some embodiments, the implant may include a second sheet (10b) surrounding the first sheet, made of the same or a different material, to form a double envelope, for example as shown in FIG. 2B. Such a double envelope may offer many different advantages, in particular for thermal insulation protecting the tissues from the heat of polymerization (for example, thanks to a fluid limiting heat transmission) or simply to provide additional safety to prevent cement leakage in the event of one of the sheets tearing. In this case, at least one of the ends of the implant, in particular the proximal end (11), may comprise an additional ring or cap concentric with the first ring or cap or, for example as shown in FIG. 2B, a double-channel ring or a double ring, to secure this second sheet (10b) while maintaining a space between it and the first sheet (10), but it is possible to secure these two sheets (10, 10b) together at the ends. In the case of two more widely spaced sheets, it is possible to provide an injection inlet between the two sheets (10, 10b) for a fluid that is different from or identical to the first, for example via a double ring or a single ring with two conduits. Such a double ring may, for example, comprise spacers between a first ring and a second ring (11b) which is concentric with the first to form between them an annular conduit allowing the injection of this second fluid (such as, for example, a lubricating fluid improving the sliding of one sheet relative to the other and thus facilitating deployment). Of course, other arrangements are possible as long as they provide a conduit opening into the envelope formed by the first sheet and a conduit opening into the space between the two sheets. These two sheets can then be folded and rolled at the same time or successively during manufacture, but their welds (or crush bonds) between them and/or to the ring and/or to the base will be made successively to ensure that the space between them is maintained. These double-sheet designs allow preforming of the injection site (by crushing the spongy bone tissue) but can also, for example, allow the fluid to be injected in two stages for better adjustment of the shape, the resulting temperature in the tissues, and/or the polymerization speed of the fluid (for example, by adjusting the mixture of cement compounds). Furthermore, as it is possible to use a second fluid other than cement inside, it is possible to use the compartment between the two sheets as a cooling circuit by circulating a fluid during the polymerization of the cement, so as to protect the tissue from the heat produced during said polymerization. Such a double-sheet implant (1) therefore requires a double cannula comprising two concentric or parallel conduits, each opening into one of the spaces provided, as will be understood by those skilled in the art from FIG. 2B without further explanation.

The term “secured” in this case means that the two elements are secured to one another, either permanently (or quasi-permanently), but also sometimes means that a connection is made so that one element can be actuated by another. Thus, screw-fastening or collaboration between shapes for temporarily locking the elements together are covered by this non-limiting term.

The terms ring, sleeve, or tube refer to hollow structures such as bands, conduits or pipes, but in a non-limiting manner, notably assuming various shapes (on the inside as on the outside), although a cylindrical shape is preferred. The term canal by contrast is preferably used herein to refer to a passage rather than to the element that contains it, and the term opening in this case refers to the fact that an element is open and able to be passed through, emerging into another structure or another element. In general, the terms sleeve, tube or conduit refer to longer elements than rings or bands, although their use herein likewise is non-limiting. Furthermore, the terms socket or cup also refer to hollow structures that are open at one end but are closed at the other end, such as plugs, closures, constrictions or restrictions, and these terms are used indiscriminately without any limitation.

It also should be noted that the fluid injection instrument (Ac) can be provided with means for controlling the pressure and/or the injected amount (a pressure gauge or at least gradations, for example) and for indicating the resulting volume, so as to effectively control the expansion into the bone tissues. Advantageously, means for controlling the injected air can be present in order to adapt the cement injection as a function of the air discharged in the hollow tubes or cannulas of the instruments (with the air generally easily escaping from the implant to the instrument by virtue of the clearance between the parts (rods and tubes or cannulas) of the instrument).

Finally, it is understood that the instrumentation proposed in the present application in some embodiments, using a relatively conventional implant holder (or ancillary) to hold the implant and introduce it into the bone tissues, but also that is less conventional for expanding it into the bone tissues, also offers the advantage that all the implantation and stabilisation steps can be carried out with a single instrument and in a continuous operation. Indeed, the ancillary with a hollow tube for conveying cement through the tube that retains the cement allows an instrument to be provided that allows the surgical intervention to be performed quickly and efficiently. After drilling, the implant is introduced and, without withdrawing the instrument, the casing can be inflated with the cement and then the tool can be withdrawn before, during or even after the polymerisation of the cement (for example, using a mechanism for cutting the hardened cement as the instrument rotates). The time taken to perform the surgical operation is clearly markedly reduced, but also the stability of the implant is improved, which implant is not released at any time until it has been stabilised by the injection of cement filling all the free volumes around it, unlike in some solutions of the prior art.

In general, the present invention relates to a system for human orthopaedic surgery for restoring the volume and/or the geometry of a bone, by expanding at least one expandable bone implant (1) between a folded configuration and a deployed configuration, the implant comprising a body extending along a longitudinal axis (L) between a proximal end (11) connectable with an implantation instrument (A) for holding the implant and a distal end (12) intended to be firstly inserted into the bone, the system being characterised in that it comprises at least one expandable bone implant (1) and at least one corresponding implantation instrument (A), selected from among a plurality of implants (1) and instruments (A) available through computing means (PC)

Certain embodiments of the present application relate to a human orthopedic surgical system for restoring the volume and/or geometry of a bone by expanding at least one expandable bone implant (1) between a collapsed configuration and an expanded configuration, comprising a body extending along a longitudinal axis (L) between a proximal end (11) capable of cooperating with an implantation instrument (A) to hold the implant and a distal end (12) intended to be inserted first into the bone, the system being characterized in that it comprises a plurality of expandable bone implants (1) and at least one implantation instrument (A) corresponding to each of said implants (1) and at least one planning aid showing said implants (1) and instrument (A), with reference to at least one standardized classification of the various types of fractures identified, in particular vertebral compression fractures, characterized in that said implants:
extend along a longitudinal axis (L) between a proximal end (11) connectable to an implantation instrument (A) for holding the implant (1) and a distal end (12) intended to be inserted first into the bone;
are deployable from a folded configuration to a deployed configuration;
are selected from:
   a) an implant comprising a hollow body whose wall is formed by a sheet (10) of a biocompatible metal alloy, sealed on itself between said proximal (11) and distal (12) ends and having, at least in the folded configuration, a plurality of pairs of folds lying on top of each other and wound around the longitudinal axis (L), said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1);
   b) an implant in which at least two faces, for example upper and lower, each comprise a plate (13, 14) for contact with bone tissue and are connected, via at least one hinge, to at least one pair of support arms (131, 141), each oriented in opposite directions toward one of said distal (11) and proximal (12) ends, the implant (1) comprising a central axis (3) or a housing capable of receiving such an axis extending through a sliding sleeve at the proximal end (11) to a traction ring or sleeve at the distal end (12) to allow said ends to be brought closer together, causing the support arms (131, 141) to pivot, causing the plates (13, 14) to move apart and the implant to expand, and either:
      at least two other sides of said implant are each covered by and secured to at least one sheet (10) of a biocompatible metal alloy in a sealed manner, said sheet (10) comprising a plurality of folds laid on top of each other in a folded configuration, said sheet being plastically deformable with a total surface area greater than or equal to the lateral surface area of the implant in the deployed configuration so as to form a sealed compartment capable of receiving a fluid inside the cavity obtained by the expansion of the implant
      an envelope formed by a sheet (10) of a biocompatible metal alloy, sealed shut on itself and enclosing said implant from the proximal end (11) to the distal end (12), said sheet (10) having a plurality of pairs of folds lying on top of each other and wound around the longitudinal axis (L) said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1);
   c) an implant (1) having at least two faces, for example upper and lower, each of which comprises at least one plate (13, 14, 15), each supported by at least two support arms (131, 141, 151), via a respective hinge, an expansion sleeve or ring (20), at least two expansion arms (132, 142, 152) each comprising a hinge connecting them to one of the ends of one of the plates (13, 14, 15) and a hinge connecting them to an expansion sleeve or ring (20) that can be moved away from a central axis (3) of the implant (1) to exert traction on the plates (13, 14, 15), via the expansion arms (132, 142, 152), causing said support arms (131, 141, 151) to pivot, causing the plates (13, 14, 15) to move apart and resulting in controlled expansion of the implant (1) between said folded configuration and said deployed configuration, and:
      this implant (1) optionally comprising a casing formed by a sheet (10) of a biocompatible metal alloy, sealed shut on itself and enclosing said implant from the proximal end (11) to the distal end (12), said sheet (10) having a plurality of pairs of folds lying on top of each other and wound around the longitudinal axis (L), said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1).

Certain embodiments of the present application relate to a human orthopedic surgery system as detailed above or elsewhere in the present application, but with a combination of implants, instruments, and planning support.

In certain embodiments, said planning support is implemented by means of computer technology (PC) comprising a human-machine interface and executing instructions on a processor enabling:
the display of information relating to at least one standardized classification of the various types of fractures identified, in particular vertebral compression fractures;
the selection of at least one type of fracture and at least one piece of information relating to the dimensions of the fractured bone, then
displaying the implants (1) and instruments (A) of the system that can be used for the planned restoration and, if necessary, instructions or advice on the procedure to be followed, for example with a display of references for the implants or various systems that can be used for each fracture identified in the classification, then
the user selecting one of these proposals and recommendations via the human-machine interface, causing the following instructions or recommendations to be displayed to provide assistance with the procedure or its planning, with the acquisition of data provided by the user on the planned surgical procedure

In some embodiments, said selection of the type of fracture in the classification is carried out either by the user or automatically by the computing means (PC) by virtue of training on fracture databases for the automatic classification thereof based on technical information and/or images provided by the user and/or previously acquired by the computing means, then correlating them with said standardised fracture classification, with the automatic selection preferably being able to be validated or modified by the user.

In some embodiments, one of the selectable implants is an expandable bone implant (1) for human orthopaedic surgery for restoring the volume and/or the geometry of a bone, by expanding between a folded configuration and a deployed configuration, said implant comprising a hollow body extending along a longitudinal axis (L) between a proximal end (11) connectablewith an implantation instrument (A) for holding the implant and a distal end (12) intended to be firstly inserted into the bone, and which is characterised in that:
the wall of said hollow body is formed by a sheet (10) made of a biocompatible metal alloy, closed on itself in a sealed manner, between said proximal (11) and distal (12) ends;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) comprises a ring sealably secured to the lying-down and rolled folds of said sheet (10) over the entire periphery of the distal end (11), with the opening passing through the ring providing an entrance to the inside of the hollow body of the implant (1);
said distal end (12) comprises a cup closing the distal end (12) and sealably secured to the lying-down and rolled folds of said sheet (10) over the entire periphery of the distal end of said hollow body;
with said sheet (10) being plastically deformable to allow the implant to expand from the folded configuration to the deployed configuration when a fluid is injected into the implant (1).

In some embodiments, one of the selectable implants is an expandable bone implant (1) for human orthopaedic surgery for restoring the volume and/or the geometry of a bone, by expanding between a folded configuration and a deployed configuration, said implant extending along a longitudinal axis (L) between a proximal end (11) connectable with an implantation instrument (A) for holding the implant and a distal end (12) intended to be firstly inserted into the bone, with at least two faces of the implant, for example, the upper and lower faces, each comprising a flange (13, 14) for making contact with the bone tissues, with each of the flanges comprising a central portion (130, 140) connected, by means of at least one hinge, to at least one pair of support arms (131, 141) each oriented in opposite directions within each pair, with one arm of each pair being connected by a hinge to the distal end (11), while the other arm is connected by a hinge to the proximal end (12), with the implant (1) being adapted to receive or comprising a central shaft (3) extending through a sliding sleeve at the proximal end (11) to a ring or traction socket at the distal end (12) where it is capable of transferring a traction force, when it is actuated by an instrument (A), to the distal end (12) in order to allow it to be moved closer to the proximal end (11), causing the support arms (131, 141) to pivot, resulting in the flanges (13, 14) moving away from each other and, consequently, the implant expanding between the folded configuration and the deployed configuration.

In some embodiments, the implant (1) is also characterised in that:
at least two other faces of the implant, between those comprising the flanges, are covered with at least one sheet (10) per face, made of a biocompatible metal alloy, and are sealably secured to the central portions (130, 140) under the flanges, to the lateral faces of the arms (131, 141) and to the lateral faces of the proximal end (11) and the distal end (12);
said sheet (10) is plastically deformable to allow the implant to expand and has, at least in the folded configuration, a plurality of antiform folds (101), called convex folds, and synform folds (102), called concave folds, with said folds lying on top of each other in the folded configuration, with the total surface area of said sheet being greater than or equal to the lateral surface area of the implant in the deployed configuration so as to form a sealed compartment adapted to receive a fluid inside the cavity obtained by the expansion of the implant.

In some embodiments, said implant (1) comprises a casing enclosing said implant from the proximal end (11) to the distal end (12) and in that:
said casing is formed by a sheet (10) made of a biocompatible metal alloy, closed on itself in a sealed manner;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) is extended by a sealing sleeve sealably secured to the lying-down and rolled folds of said sheet (10) over the entire periphery of the proximal end (11);
said distal end (12) is extended by a socket sealably secured to the lying-down and rolled folds of said sheet (10) over the entire periphery of the distal end (12) of said implant (1);
said sheet (10) is plastically deformable to allow the implant to expand from the folded configuration to the deployed configuration, forming a sealed casing enclosing the implant and allowing any leakage to be avoided when a fluid is injected into the implant (1) and the casing.

In some embodiments, one of the selectable implants is an expandable bone implant (1) for human orthopaedic surgery for restoring the volume and/or geometry of a bone by expanding between a folded configuration and a deployed configuration, said implant comprising a central shaft (3) and extending along a longitudinal axis (L) between a proximal end (11) connectable with an implantation instrument (A) for holding the implant and a distal end (12) intended to be firstly inserted into the bone, with at least two faces, for example, an upper and a lower face, of the implant each comprising at least one flange (13, 14, 15) for making contact with the bone tissues, with each of the flanges being supported by at least two support arms (131, 141, 151) each, by means of a hinge on the central shaft (3) and a hinge under the respective flange (13, 14, 15) of each of said support arms (131, 141, 151), characterized in that:
an expansion ring or socket (20) is arranged on the same axis as said central shaft (3);
at least two expansion arms (132, 142, 152) each comprise a hinge connecting them to one of the ends of one of the flanges (13, 14, 15) and a hinge connecting them to said expansion ring or socket (20);
said expansion ring or socket (20) and the proximal end of the central shaft (3) are adapted to engage, respectively or vice versa, with a hollow tube (A1) for gripping the implant (1) of an implantation instrument (A) and with an expansion rod (A3) of said instrument (A);
said expansion rod (A3) is adapted to slide inside said hollow tube (A1) so as to cause separation between said socket (3) and said central shaft (3), applying a traction force to the flanges (13, 14, 15), via expansion arms (132, 142, 152), thereby causing said support arms (131, 141, 151) to pivot, thus resulting in the flanges (13, 14, 15) moving away from the central shaft (3), resulting in controlled expansion of the implant (1) between said folded configuration and said deployed configuration.

In some embodiments, the implant (1) comprises a casing enclosing said implant from the proximal end (11) to the distal end (12) and in that:
said casing is formed by a sheet (10) made of a biocompatible metal alloy, closed on itself in a sealed manner;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) is extended by a sealing sleeve sealably secured to the lying-down and rolled folds of said sheet (10) over the entire periphery of the proximal end (11);
said distal end (12) is extended by a socket sealably secured to the lying-down and rolled folds of said sheet (10) over the entire periphery of the distal end (12) of said implant (1);
said sheet (10) is plastically deformable to allow the implant to be expanded from the folded configuration to the deployed configuration, forming a sealed casing enclosing the implant and allowing any leakage to be avoided when a fluid is injected into the implant (1) and the casing.

The present application also relates to a method for planning the implantation of an implant system as described above, for restoring the volume and/or the geometry of a bone, by expanding between a folded configuration and a deployed configuration, characterised in that it comprises automatic selection, and/or selection by the practitioner performing the implantation, of at least one type of fracture identified in at least one standardised classification, via computing means (PC) and presented to said practitioner, then accessing, following this selection, at least one implant system compatible with said type of fracture and at least one operating protocol comprising a series of steps to be performed as a function of said selection.

In some embodiments, the method is implemented by computing means executing instructions on a processor allowing:
information to be displayed relating to at least one classification of the types of fractures identified in traumatology;
the selection of at least one type of fracture and at least one item of information relating to the dimensions of the fractured bone; then
instructions or recommendations to be displayed concerning the procedure to be followed and the various systems that can be used for the procedure, for example, displaying references for the implants or the various systems that can be used for each fracture identified in the classification; then
the selection of one of these recommendations, causing the following instructions or recommendations to be displayed in order to provide said assistance step-by-step.

In some embodiments, displaying instructions or recommendations involves displaying selectable functions for selecting from among actions to be carried out in order to continue the procedure and/or from among implants to be selected as a function of the operating protocol selected as a function of the type of fracture identified according to said classification, allowing the practitioner to validate a selection and optionally allowing predefined options to be proposed in the validated protocol, such as, for example, acquiring X-rays at the end of certain implemented steps.

In some embodiments, said selectable functions include functions allowing the practitioner to modify said protocol and generate the display of a protocol modification window, either for selecting a protocol from among other possible protocols for said type of fracture identified according to the classification, or for creating a customised operating protocol for the fracture that is being repaired.

In some embodiments, said selectable functions allow the operations and operating protocols, notably customised ones, to be recorded that are used by various practitioners and their associations with fractures, with the optional recording of customised protocols created by practitioners, in order to subsequently propose them.

In some embodiments, the method comprises:
detecting fractures by means of an artificial intelligence identification system, using X-rays/scans/MRIs loaded into the software and by recognising defects and/or differences from the supposed reality, the supposed fracture zone is identified, in order to facilitate the identification of a specific feature for the practitioner. The database is gradually enriched taking into account the choices of the practitioner.

Some embodiments anticipate recognising the type of fracture involving identification according to the OA (Orthopaedic Association) Classification or any other classification stored in the computing means (PC). The software can then classify the type, the severity, and the position of the fracture, defined, for example, by:
the Zone: Distal/Median/Proximal, described as wedge-shaped, biconcave, or a slice;
the Severity: Normal vertebra/Slight fracture/Moderate fracture/Severe fracture.

Preferably, a bone densitometry test is then performed so that the software can analyse and compare with a model in order to establish the direct and future risks of the fracture.

Furthermore, based on the identification and the level of the fracture according to the reduction to be carried out, the software can propose the type of implant and protocol best suited to the morphology/bone density, to the type, and to the severity of the fracture. The most suitable implant is generally selected as a function of the type of correction: and preferably the software proposes a recommendation, but the practitioner must validate and/or can modify the recommendation of the software.

Some embodiments provide assistance during the operation involving the compliance of the protocol selected by the practitioner and/or corrected by the practitioner being checked, with an increase and/or a reduction in the corrected volume.

Furthermore, in some embodiments, the system comprises at least one instrument (A) extending along a longitudinal axis (L) between a proximal end (11) adapted to engage with said implantation instrument (A) for holding the implant and a distal end (12) intended to be firstly inserted into the bone, said instrument (A) comprising a main part that can be gripped by a practitioner, an implant-holding portion (AA) comprising a gripping tube (A0) extending along the longitudinal axis (L) and having, at its distal end, retention means that complement engagement means for engaging the implant, for holding the implant by its proximal end (11), said instrument (A) being characterised in that it comprises an instrument (Ac) for injecting fluid, such as bone cement, into said implant (1), comprising at least one cavity adapted to receive the fluid, said injection instrument (Ac) being disposed behind the implant-holding portion (AA) along the longitudinal axis (L) and comprising at least one cannula (A0, A1, A3) for conveying the fluid to the inside of the implant (1) while it is still held by said implant-holding portion (AA).

In some embodiments, the instrument (A) also comprises an instrument (Ae) for expanding the implant (1) adapted to engage with mechanical means for expanding the implant (1), by virtue of an expansion rod (A3) passing through said implant-holding portion (AA) and said gripping tube (A0) in order to actuate said mechanical expansion means. In some embodiments, said expansion rod (A3) passes through said injection instrument (Ac) to the distal end of the gripping tube (AO).

The present application describes various technical features and advantageous with reference to the figures and/or to various embodiments. A person skilled in the art will appreciate that the technical features of a given embodiment actually can be combined with features of another embodiment unless otherwise explicitly stated or unless it is obvious that these features are incompatible or that combining them would not provide a solution to at least one of the technical problems cited in the present application. In addition, the technical features described in a given embodiment can be taken in isolation from the other features of this embodiment unless otherwise explicitly stated.

### DETAILED LIST OF REFERENCES IN THE FIGURES

1 implant
10 sheet
11 proximal end
101 antiform fold
102 synform fold
110 proximal weld
12 distal end
120 distal weld (fluidtight connection)
121 compression fixing (for example, split ring)
3 central shaft
31 central-shaft conduit
32 openings in the central-shaft conduit
13 first flange
14 second flange
15 third flange
20 expansion ring
131 first-flange support arm
141 second-flange support arm
151 third-flange support arm
132 first-flange expansion arm
142 second-flange expansion arm
152 third-flange expansion arm
130 first-flange central support arm
140 second-flange central support arm
150 third-flange central support arm
A implantation instrument
Ac fluid-injection instrument
A0 hollow gripping tube
A1 injection cannula
A3 expansion rod
V vertebra
VCF vertebral compression fracture

1. A system for human orthopaedic surgery for restoring the volume and/or the geometry of a bone, by expanding at least one expandable bone implant (1) between a folded configuration and a deployed configuration, the implant comprising a body extending along a longitudinal axis (L) between a proximal end (11) adapted to engage with an implantation instrument (A) for holding the implant and a distal end (12) configured to be firstly inserted into the bone, the system comprising a plurality of expandable bone implants (1) and at least one (1) and at least one implantation instrument (A) corresponding to each of said implants (1), with reference to at least one standardized classification of the various types of fractures identified, in particular vertebral compression fractures, wherein said implants:
extend along a longitudinal axis (L) between a proximal end (11) connectable to an implantation instrument (A) for holding the implant (1) and a distal end (12) intended to be inserted first into the bone;
are deployable from a folded configuration to a deployed configuration;
are selected from:
d) an implant comprising a hollow body whose wall is formed by a sheet (10) of a biocompatible metal alloy, sealed on itself between said proximal (11) and distal (12) ends and having, at least in the folded configuration, a plurality of pairs of folds lying on top of each other and wound around the longitudinal axis (L), said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1);
e) an implant in which at least two faces, each comprise a plate (13, 14) for contact with bone tissue and are connected, via at least one hinge, to at least one pair of support arms (131, 141) each oriented in opposite directions toward one of said distal (11) and proximal (12) ends, the implant (1) comprising a central axis (3) or a housing capable of receiving such an axis extending through a sliding sleeve at the proximal end (11) to a traction ring or sleeve at the distal end (12) to allow the said ends to be brought closer together, causing the support arms (131, 141) to pivot, causing the plates (13, 14) to move apart and the implant to expand, and either:
at least two other faces of said implant are each covered by and secured to at least one sheet (10) of a biocompatible metal alloy in a sealed manner, said sheet (10) comprising a plurality of folds laid one on top of the other in a folded configuration, said sheet being plastically deformable with a total surface area greater than or equal to the lateral surface area of the implant in the deployed configuration so as to form a sealed compartment capable of receiving a fluid inside the cavity obtained by the expansion of the implant;
an envelope formed by a sheet (10) of a biocompatible metal alloy, sealed shut on itself and enclosing said implant from the proximal end (11) to the distal end (12), said sheet (10) having a plurality of pairs of folds lying on top of each other and wound around the longitudinal axis (L) said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1);
f) an implant (1) having at least two faces, each comprising at least one plate (13, 14, 15), each supported by at least two support arms (131, 141, 151) via a respective hinge, an expansion sleeve or ring (20), at least two expansion arms (132, 142, 152) each comprising a hinge connecting them to one of the ends of one of the plates (13, 14, 15) and a hinge connecting them to an expansion sleeve or ring (20) that can be moved away from a central axis

(3) of the implant (1) to exert traction on the plates (13, 14, 15), via the expansion arms (132, 142, 152), causing said support arms (131, 141, 151) to pivot, causing the plates (13, 14, 15) to move apart and resulting in controlled expansion of the implant (1) between said folded configuration and said deployed configuration, and:
this implant (1) optionally comprising a casing formed by a sheet (10) of a biocompatible metal alloy, sealed shut on itself and enclosing said implant from the proximal end (11) to the distal end (12), said sheet (10) having a plurality of pairs of folds lying on top of each other and wound around the longitudinal axis (L), said proximal (11) and distal (12) ends comprising, respectively, a ring providing an entrance to the interior of the hollow body and a base closing the hollow body, said ring and said base being sealed to the folds lying on top of each other and wound around said sheet (10) over the entire periphery of their respective proximal (11) or distal (12) ends, said sheet (10) being plastically deformable to allow the implant to expand from the folded configuration to the deployed configuration when a fluid is injected into the implant (1).

2. The system according to claim 1, wherein said planning support is implemented by means of computer equipment (PC) comprising a human-machine interface and executing instructions on a processor enabling:
the display of information relating to said standardized classification of the various types of fractures identified, in particular vertebral compression fractures;
the selection of at least one type of fracture and at least one piece of information relating to the dimensions of the fractured bone, then
displaying the implants (1) and instruments (A) of the system that can be used for the planned restoration and, where applicable, instructions or advice on the procedure to be followed, then
the user selecting one of these proposals and recommendations via the human-machine interface, causing the following instructions or recommendations to be displayed to provide assistance with the procedure or its planning, with the acquisition of data provided by the user on the planned surgical procedure.

3. The system according to claim 2, wherein said selection of the type of fracture in the classification is carried out either by the user or automatically by the computing means (PC) by virtue of training on fracture databases for the automatic classification thereof based on technical information and/or images provided by the user and/or previously acquired by the computing means, then correlating them with said standardised fracture classification.

4. The system according to claim 1, wherein one of the selectable implants is an expandable bone implant (1) for human orthopaedic surgery for restoring the volume and/or the geometry of a bone, by expanding between a folded configuration and a deployed configuration, said implant comprising a hollow body extending along a longitudinal axis (L) between a proximal end (11) connectable with an implantation instrument (A) for holding the implant and a distal end (12) intended to be firstly inserted into the bone, and wherein:
the wall of said hollow body is formed by a sheet (10) made of a biocompatible metal alloy, closed on itself in a sealed manner, between said proximal (11) and distal (12) ends;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) comprises a ring sealably secured to the lying-down and rolled folds of said sheet (10) over the entire periphery of the distal end (11), with the opening passing through the ring providing an entrance to the inside of the hollow body of the implant (1);
said distal end (12) comprises a cup closing the distal end (12) and sealably secured to the lying-down and rolled folds of said sheet (10) over the entire periphery of the distal end of said hollow body; and
with said sheet (10) being plastically deformable to allow the implant to expand from the folded configuration to the deployed configuration when a fluid is injected into the implant (1).

5. The system according to claim 1, characterised in that one of the selectable implants is an expandable bone implant (1) for human orthopaedic surgery for restoring the volume and/or the geometry of a bone, by expanding between a folded configuration and a deployed configuration, said implant extending along a longitudinal axis (L) between a proximal end (11) connectable with an implantation instrument (A) for holding the implant and a distal end (12) intended to be firstly inserted into the bone, with at least two faces of the implant, for example, the upper and lower faces, each comprising a flange (13, 14) for making contact with the bone tissues, with each of the flanges comprising a central portion (130, 140) connected, by means of at least one hinge, to at least one pair of support arms (131, 141) each oriented in opposite directions within each pair, with one arm of each pair being connected by a hinge to the distal end (11), while the other arm is connected by a hinge to the proximal end (12), with the implant (1) being adapted to receive or comprising a central shaft (3) extending through a sliding sleeve at the proximal end (11) to a ring or traction socket at the distal end (12) where it is capable of transferring a traction force, when it is actuated by an instrument (A), to the distal end (12) in order to allow it to be moved closer to the proximal end (11), causing the support arms (131, 141) to pivot, resulting in the flanges (13, 14) moving away from each other and, consequently, the implant expanding between the folded configuration and the deployed configuration.

6. The system according to claim 3, wherein the implant (1) further comprises:
at least two other faces of the implant, between those comprising the flanges, are covered with at least one sheet (10) per face, made of a biocompatible metal alloy, and are sealably secured to the central portions (130, 140) under the flanges, to the lateral faces of the arms (131, 141) and to the lateral faces of the proximal end (11) and the distal end (12);
said sheet (10) is plastically deformable to allow the implant to expand and has, at least in the folded configuration, a plurality of antiform folds (101), called convex folds, and synform folds (102), called concave folds, with said folds lying on top of each other in the folded configuration, with the total surface area of said sheet being greater than or equal to the lateral surface area of the implant in the deployed configuration so as to form a sealed compartment adapted to receive a fluid inside the cavity obtained by the expansion of the implant.

7. The system according to claim 3, wherein said implant (1) comprises a casing enclosing said implant from the proximal end (11) to the distal end (12) and wherein:
said casing is formed by a sheet (10) made of a biocompatible metal alloy, closed on itself in a sealed manner;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) is extended by a sealing sleeve sealably secured to the lying-down and rolled folds of said sheet (10) over the entire periphery of the proximal end (11);
said distal end (12) is extended by a socket sealably secured to the lying-down and rolled folds of said sheet (10) over the entire periphery of the distal end (12) of said implant (1); and
said sheet (10) is plastically deformable to allow the implant to expand from the folded configuration to the deployed configuration, forming a sealed casing enclosing the implant and allowing any leakage to be avoided when a fluid is injected into the implant (1) and the casing.

8. The system according to claim 1, wherein one of the selectable implants is an expandable bone implant (1) for human orthopaedic surgery for restoring the volume and/or the geometry of a bone, by expanding between a folded configuration and a deployed configuration, with said implant comprising a central shaft (3) and extending along a longitudinal axis (L) between a proximal end (11) connectable with an implantation instrument (A) for holding the implant and a distal end (12) intended to be firstly inserted into the bone, with at least two faces of the implant each comprising at least one flange (13, 14, 15) for making contact with the bone tissues, with each of the flanges being supported by at least two support arms (131, 141, 151) each, by means of a hinge on the central shaft (3) and a hinge under the respective flange (13, 14, 15) of each of said support arms (131, 141, 151), wherein:
an expansion ring or socket (20) is arranged on the same axis as said central shaft (3);
at least two expansion arms (132, 142, 152) each comprise a hinge connecting them to one of the ends of one of the flanges (13, 14, 15) and a hinge connecting them to said expansion ring or socket (20);
said expansion ring or socket (20) and the proximal end of the central shaft (3) are adapted to engage, respectively or vice versa, with a hollow tube (A1) for gripping the implant (1) of an implantation instrument (A) and with an expansion rod (A3) of said instrument (A); and
said expansion rod (A3) is adapted to slide inside said hollow tube (A1) so as to cause separation between said socket (3) and said central shaft (3), applying a traction force to the flanges (13, 14, 15), via expansion arms (132, 142, 152), thereby causing said support arms (131, 141, 151) to pivot, thus resulting in the flanges (13, 14, 15) moving away from the central shaft (3), resulting in controlled expansion of the implant (1) between said folded configuration and said deployed configuration.

9. The system according to claim 6, wherein the implant (1) comprises a casing enclosing said implant from the proximal end (11) to the distal end (12) and wherein:
said casing is formed by a sheet (10) made of a biocompatible metal alloy, closed on itself in a sealed manner;
said sheet (10) has, at least in the folded configuration, a plurality of pairs of folds, with each of the pairs comprising an antiform fold (101), called convex fold, and a synform fold (102), called concave fold, with said folds lying on top of each other in a folded configuration so that the surfaces present between each of said convex and concave folds are rolled around the longitudinal axis (L);
said proximal end (11) is extended by a sealing sleeve sealably secured to the lying-down and rolled folds of said sheet (10) over the entire periphery of the proximal end (11);
said distal end (12) is extended by a socket sealably secured to the lying-down and rolled folds of said sheet (10) over the entire periphery of the distal end (12) of said implant (1); and
said sheet (10) is plastically deformable to allow the implant to be expanded from the folded configuration to the deployed configuration, forming a sealed casing enclosing the implant and allowing any leakage to be avoided when a fluid is injected into the implant (1) and the casing.

10. A method for planning the implantation of an implant system according to claim 1, for restoring the volume and/or the geometry of a bone, by expanding between a folded configuration and a deployed configuration, wherein the method comprises automatic selection, and/or selection by the practitioner performing the implantation, of at least one type of fracture identified in at least one standardised classification, via computing means (PC) and presented to said practitioner, then accessing, following this selection, at least one implant system compatible with said type of fracture and at least one operating protocol comprising a series of steps to be performed as a function of said

11. The method according to claim 10, wherein the method is implemented by computing means executing instructions on a processor allowing:
information to be displayed relating to at least one classification of the types of fractures identified in traumatology;
the selection of at least one type of fracture and at least one item of information relating to the dimensions of the fractured bone; then
instructions or recommendations to be displayed concerning the procedure to be followed and the various systems that can be used for the procedure; then
the selection of one of these recommendations, causing the following instructions or recommendations to be displayed in order to provide said assistance step-by-step.

12. The method according to claim 11, wherein displaying instructions or recommendations involves displaying selectable functions for selecting from among actions to be carried out in order to continue the procedure and/or from among implants to be selected as a function of the operating protocol selected as a function of the type of fracture identified according to said classification, allowing the practitioner to validate a selection and optionally allowing predefined options to be proposed in the validated protocol.

13. The method according to claim 12, wherein said selectable functions include functions allowing the practitioner to modify said protocol and generate the display of a protocol modification window, either for selecting a protocol from among other possible protocols for said type of fracture identified according to the classification, or for creating a customised operating protocol for the fracture that is being repaired.

14. The method according to claim 13, wherein said selectable functions allow the operations and operating protocols, notably customised ones, to be recorded that are used by various practitioners and their associations with fractures, with the optional recording of customised protocols created by practitioners, in order to subsequently propose them.
